(19) United States
(12) Patent Application Publication
(10) Pub. No.: US 2006/0248606 A1
(43) Pub. Date: Nov. 2, 2006

(54) Mouse model for hepatitis C

(76) Inventor: Jeffrey S. Glenn, Palo Alto, CA (US)

Correspondence Address: MORRISON & FOERSTER LLP, 12531 HIGH BLUFF DRIVE, SUITE 100, SAN DIEGO, CA, 92130-2040, US

(21) Appl. No.: 11/480,012

(22) Filed: Jun. 29, 2006

Related U.S. Application Data
(63) Continuation of application No. 10/157,099, filed on May 28, 2002.
(60) Provisional application No. 60/293,750, filed on May 25, 2001.

(51) Int. Cl. A01K 67/027; A61K 48/00

(52) U.S. Cl. 800/14; 800/18; 424/93.2

(57) ABSTRACT

A rodent model for HCV infection is obtained by introducing into rodents which harbor a liver-specific defect an expression system which comprises an HCV replicon or a reverse transcript thereof and at least a nucleotide sequence encoding a rescue protein which remedies the defect.

### CROSS-REFERENCE TO RELATED APPLICATIONS

This application is a continuation of of U.S. patent application Ser. No. 10/157,099, filed May 28, 2002, which claims the benefit of U.S. provisional patent application Ser. No. 60/293,750 filed May 25, 2001, the disclosures of which are herein incorporated by reference in their entirety.

### TECHNICAL FIELD

This application is The invention concerns a rodent model for hepatitis C infection (HCV). Specifically, hepatitis C can be successfully replicated in rodents, especially mice, through the use of an organ-specific selectable marker. In one preferred approach, HCV replicons which express a liver-specific selectable marker are introduced into hepatocytes and the hepatocytes implanted into the liver of an appropriate “knockout” mouse.

### BACKGROUND ART

Hepatitis C virus (HCV) infects almost 200 million people worldwide and is a debilitating and often eventually fatal infection. Current therapies remain inadequate as they are expensive, plagued with side effects, and are, in general, not very effective. Accordingly, the need for a safe and effective treatment, both prophylactic and therapeutic, is needed.

The search for such treatments has been hampered by the lack of an adequate animal model. Currently, attempts to mimic the progress of infection in non-human organisms have been confined to chimpanzees. It has not been possible to construct a more useful, convenient, and more ethically acceptable murine model because it has not been possible to induce HCV infection in mice, for example, using patient serum. Human hepatocytes can be infected by HCV, and while it is possible to engraft murine hepatocytes into a mouse recipient liver, it has not been possible to maintain, efficiently, human hepatocytes engrafted in murine liver.

It has now been found that HCV can be produced in rodents, preferably mice, by introducing into mice that harbor a liver defect, an expression system which results in HCV replication as well as the production of a protein that is able to rescue the animal from the liver defect. In one approach, a DNA-based vector can be introduced directly into the murine liver; in another approach, HCV replicons can be transfected into murine or other rodent hepatocytes using a selectable marker. By using hepatocytes derived from the same species, the transplant engraftment problem is obviated. By using a selectable marker, successful maintenance of HCV replication in both hepatocytes and in the animal is achieved, and HCV-harboring liver cells are maintained in the animal.

Selectable markers in whole animals repair metabolic deficiencies in these animals. A multiplicity of “knockout” mice and other rodents which lack certain essential enzymes or proteins have been constructed. One such mouse, particularly useful in the method of the present invention is that wherein the fumarylacetoacetate hydrolase (FAH) enzyme is not produced. FAH is required for the last step in tyrosine catabolism. FAH knockout mice die shortly after birth from hepatic dysfunction because they are unable to eliminate the catabolytes of tyrosine. It has been shown that if the flow of catabolites from tyrosine is blocked at an upstream step, for example by administering the drug 2-(2-nitro-4-trifluoromethylbenzoyl)-1,3-cyclohexane dione (NTBC) the mice can survive. It has further been shown that transplantation of as few as 1,000 wildtype FAH producing hepatocytes into FAH knockout mice can repopulate over 90% of the host liver and the mice survive even without NTBC. See Grompe, M., et al., Genes Dev. (1993) 7:2298-2307; Overturf, K., et al., I. Nat. Genet. (1996) 12:266-273.

Also helpful in the method of the invention is the ability of HCV replicons to adapt to cells they infect by sustaining one or more mutations which aid replication efficiency. This discovery was reported by Blight, K. J., et al., Science (2001) 290:1972-1974. This report describes the isolation and sequencing of replicon RNA from individual colonies produced during the course of work described by Lohman, V., et al., Science (1999) 285:110-113. This work employed a bicistronic subgenomic replicon of HCV containing the neomycin resistance gene in place of the structural genes normally contained in the virus. A comparison of the HCV genome (which is RNA) with the construct of Lohman, et al., is shown in FIG. 1.

FIG. 1A shows the native HCV genome and FIG. 1B shows the modified replicon. In Lohman's work, the human liver tumor derived cell line, Huh-7, was modified to contain the altered replicon and transformed cells were selected by G418. However, only about 1 in 1,000,000 electroporated cells gave rise to a colony. In the paper by Blight, et al., the RNA retrieved from such colonies showed adaptive mutations, and by incorporating these adaptive mutations into the original replicon of Lohman and repeating Lohman's experiment, up to four logs increase in colony formation efficiency was observed—i.e., rather than one colony per million electroporated cells, 2,000-100,000 colonies were observed per million cells. Thus, the replicons with adaptive mutations such as those described by Blight, et al., are particularly useful in the methods of the invention.

### DISCLOSURE OF THE INVENTION

The invention is directed to the construction and use of a rodent model for HCV infection. The model utilizes rodents that harbor at least one liver-specific defect, for example that are deficient in an essential hepatic protein, and is constructed by introducing into such rodents nucleic acids comprising expression systems operable in hepatocytes, wherein said systems comprise control sequences operably linked to an HCV replicon or its reverse transcript and to a nucleotide sequence encoding at least one protein that overcomes the defect. Only rodents into which the nucleic acid has been successfully introduced survive and they can then serve as models for HCV infection. These model systems then can be used to follow the progress of the infection as well as tools for evaluation and development of HCV vaccines and HCV therapies.

Thus, in one aspect, the invention is directed to a method to obtain a rodent subject which sustains HCV replication, which method comprises introducing into a rodent which exhibits a liver-specific defect a nucleic acid containing an expression system which comprises an HCV replicon or its reverse transcript and a nucleotide sequence encoding at least one protein which can overcome the defect. The expression system may also include a reporter sequence such as green fluorescent protein (GFP) or luciferase for assessing the level of the production of the rescue protein or of the production of one or more viral proteins or portions included in the expression system. In one embodiment, the invention method comprises directly administering the nucleic acids to the liver of the rodent, in which case DNA-based vectors are preferred, although RNA-based vectors can also be used. In an alternative approach, the expression system is introduced by transplanting, into a rodent, hepatocytes which are of the same species as the rodent and which have been modified to contain an expression system comprising an HCV replicon or its reverse transcript and a nucleotide sequence or its complement encoding at least one protein that overcomes a liver-specific defect in the hepatocytes and in the rodent, such as lack of an essential hepatic protein. Both hepatocytes and the rodent will have been modified to contain a liver-specific defect, for example to lack an essential hepatic protein.

As will further be described below, “reverse transcript” of the replicon may either be a cDNA transcript obtained as a product of reverse transcriptase or may be the RNA complement of the replicon, mimicking the manner in which HCV replicates.

In other aspects, the invention is directed to the rodent model system produced, to methods to use this system to evaluate HCV treatments and to follow the course of HCV infection and to methods and materials for construction of the RNA and DNA vectors and the hepatocytes for transplantation.

### BRIEF DESCRIPTION OF THE DRAWINGS

FIGS. 1A and 1B show a comparison of the native HCV genome (FIG. 1A) and a modified HCV replicon which is a template for that useful in the invention (FIG. 1B).

FIGS. 2A-2C show a comparison of the Lohman modified replicon (FIG. 2A) with the replicon containing the rescue protein-encoding nucleotide sequence (FAH) of the invention (FIG. 2B) and with the replicon of FIG. 2B which has further been modified to encode luciferase as a reporter protein fused to the selectable marker (FIG. 2C). The circle in the nonstructural protein portion of the replicon indicates an adaptive mutation site.

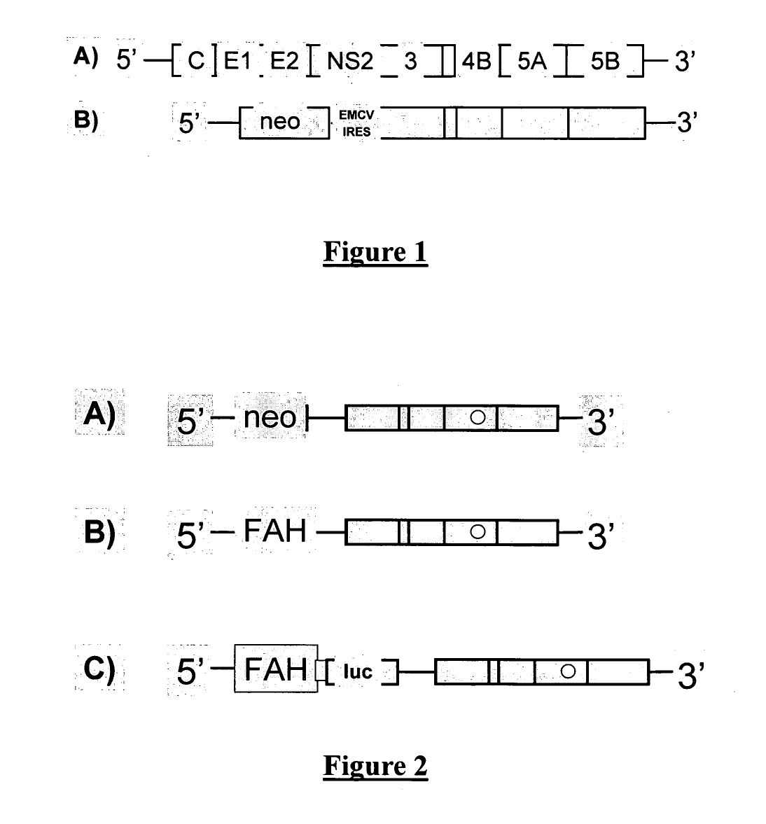

### MODES OF CARRYING OUT THE INVENTION

The invention employs standard techniques of recombinant biotechnology, genetic manipulation of transgenic animals, transplantation, and manipulation of rodent models. In one embodiment, the subjects which are to be converted into models of HCV infection are “knockout” mice or other rodents which have been genetically modified to create a deficiency in at least one enzyme or protein essential for adequate liver function. One such illustrative enzyme is FAH, described above and FAH knockout mice have been described. Other essential hepatic proteins can also serve as selectable markers in the invention and rodent, especially murine systems deficient in these enzymes can be created using standard techniques. Also useful in the invention are any spontaneously mutated mice or other rodents that may be so deficient.

By an “essential hepatic protein” is meant a protein that must be present in the liver in order for the rodent to survive or to maintain general well being. While some essential hepatic proteins which are preferred are those where the absence of such protein, e.g. an enzyme, would result in inability of the rodent to survive, it is also possible to use embodiments where the absence of the essential hepatic protein, e.g. enzyme, merely produces rodents that are distinguishable from rodents which contain the enzyme by virtue of their morbidity. If the defect is such that it is fatal, there must be a means to at least reverse the deficiency long enough to allow the subjects to remain viable long enough to become models. Some such defects can be reversed by administration of drugs; drugs are administered for the desired time period and administration is withdrawn at the time selection is desired. Especially preferred are proteins, such as FAH, whose effects can be controlled by drugs or specific compounds so that the animals survive to a suitable age for transplantation. Examples of such essential hepatic proteins include, in addition to FAH, cytochrome p450, enzymes of the glycolytic pathway, and the like.

The use of such drug-controlled deficiency is especially advantageous in that the timing involved in the selection can be varied. For instance, in the case of FAH deficient rescue by NTBC, the drug can be withdrawn before, at the time of, or just after, modification to contain the expression system for the rescue protein, FAH. The level of FAH required can be down regulated by control of re-administration and dosage of the NTBC. By controlling the protocol, waves of selection can be induced.

The genetic modification that produces a liver-specific defect need not be the knockout of an essential hepatic protein, but rather may induce a negative effect by generating a toxin or by producing an enzyme that converts an administered “pro” drug to a toxic form. The genetic modification, whether a “knockout” of an essential hepatic protein or acquisition of the ability to produce a toxin or toxin-generating enzyme, may be hemizygous if the unmodified rodent has a growth advantage over the rodent with a hemizygous modification. It may also be homozygous if the animal can survive until an age of liver cell transplant (approximately 4 weeks) either because of the inherent nature of the defect, or because the defect may be temporarily rescued by drugs (as is the case with FAH knockouts) or if the defect is such that it exhibits age-specific induction.

As further described herein, the invention employs expression systems involving an HCV replicon, at least one rescue protein and, optionally, a reporter. It should be noted that determination of the levels of a rescue protein produced, or a viral protein produced, can be used to assess the level of HCV replication, and in that sense these proteins themselves might be considered reporters. However, as used herein, the word “reporter” protein or a gene encoding a “reporter” protein refers to an extraneous protein whose production is readily detected. Examples of this protein include luciferase and the class of proteins loosely referred to as green fluorescent protein (although not all of them are green) that are readily observed. Other proteins that are easily assessed and offer some experimental convenience could also be used.

The expression system itself can assume a variety of forms. In its simplest and most straightforward form, it is an RNA which comprises an “HCV replicon” (which may either be the complete HCV genome or a sufficient portion thereof to effect replication) coupled to a nucleotide sequence encoding a rescue protein, and optionally to a nucleotide sequence encoding a reporter. In this embodiment, all of the nucleotide sequence are RNA and can be used as such. Alternatively, the complement of this entire expression system could be employed so as to mimic the manner of HCV replication and infection. In this case, the reverse RNA transcript of the HCV replicon or portion will be coupled to the complement of the nucleotide sequence encoding the rescue protein and, optionally the reporter. In still another alternative, the cDNA reverse transcript of the replicon, which comprises the complete HCV genome or a portion thereof can be used. As the cDNA constructs will be double-stranded, they will contain both the reverse transcript of the HCV replicon and the nucleotide sequence encoding the rescue protein. However, if the expression system is bicistronic, the same strand of DNA should include the reverse transcript of the replicon and the complement of the encoding nucleotide sequences. Those of skill in the art will recognize the alternative constructs and methods for their synthesis.

Depending on the nature of the deficiency of the subject, a suitable expression system is constructed wherein an HCV replicon and a nucleotide sequence encoding protein or proteins which rescue the defect are expressed. If the liver defect is a deficiency in an essential hepatic protein, the rescue protein or proteins will comprise a replacement for the essential hepatic protein. If the deficiency is a result of the production of a toxin, a protein which counteracts or which generates metabolic events which counteract the effects of the toxin may be included. If the defect is induced by an enzyme that activates an administered pro drug to form a toxic drug, the deficiency-curing protein or proteins may generate inhibitors to this enzyme or otherwise counteract the downstream effects. Also, optionally, the expression system may include a reporter gene so that the replication of HCV or the production of the rescue protein can be observed conveniently. Suitable reporters include, for example, luciferase and GFP. Bicistronic systems wherein both the HCV replicon and the rescue protein encoding sequence are operably linked to the same control sequences are preferred; however, it is possible in some instances to employ expression systems wherein each element has its own control sequences to effect expression. However, the results are believed to be more reliable in the case of bicistronic expression systems. If a reporter is used, a tricistronic system may be employed or the reporter may be fused to the marker or to the replicon.

In general, two types of expression systems can be employed. One, which is RNA-based, comprises the HCV replicon, typically modified in a manner analogous to the modified replicon of Lohman, et al., described above, where the neomycin-encoding sequence is replaced by a nucleotide sequence encoding the rescue protein. As stated above, the complement of this entire RNA, constructed as an RNA vector could also be used. Alternatively, a DNA-based expression system can be used where the HCV replicon is replaced by its reverse transcript and the double-stranded DNA encoding the rescue protein replaces the RNA nucleotide sequence encoding it in the RNA system. The expression systems may also comprise, and preferably do comprise, adaptive mutation(s) that enhance replication efficiency.

By “HCV replicon” is meant a nucleotide sequence which self-replicates using the natural replication signals of HCV and which encodes sufficient portions of the HCV protein to recapitulate at least some aspects of the natural infection. HCV itself is a 9.6 kb single-stranded RNA that encodes a single approximately 3,000 amino acid polyprotein that is proteolytically processed into structural and non-structural elements. The replicon contained in the RNA-based expression system will be an RNA transcript of at least a portion of the HCV RNA sufficient for self-replication. For example, as described in copending applications U.S. provisional applications 60/288,687 filed 3 May 2001, and 60/316,805 filed 31 Aug. 2001, and PCT application PCT US 02/13951 filed 3 May 2002, and incorporated herein by reference, certain non-structural proteins, such as NS5A, comprise amphipathic helices which associate with cytoplasmic membranes and can serve as model systems for at least a portion of the steps in HCV infection such that the effects of various treatments can be evaluated. Thus, the HCV replicon may constitute the complete HCV virion or its reverse transcripts, only the nonstructural proteins, or may be only a sufficient portion thereof, such as that including a protein encoding amphipathic helix associated with the NS5A protein which is sufficient to provide a model of at least a portion of the course of HCV infection. In some cases a helper expression system may be required.

In the DNA-based expression system, the HCV replicon which may be a complete or partial HCV genome as described above, is replaced by its reverse transcript. In this case, the expression system will require additional control sequences, including at least one promoter, as further described below.

A preferred embodiment employs a high efficiency subgenomic replicon of HCV which is described by Blight, et al., supra, by replacing the neomycin-encoding region with a nucleotide sequence encoding the rescue protein. While the adaptation described by Blight has been established in human liver tumor cell lines, the same adaptation is useful in rodent-derived hepatocytes as well, or, if necessary, HCV replicons can be adapted to rodent-based hepatocytes in culture using the techniques described by Blight, et al.

The portion of the expression system which encodes the selectable marker will be determined by the nature of the rodent into which the hepatocytes are to be transplanted. The nucleotide sequence will encode the essential hepatic protein which is deficient in the rodent or a protein which otherwise remedies a liver-specific defect.

With respect to the DNA expression system, this may be contained on a vector which is maintained as an extrachromosomal element, or may be designed to be integrated into the chromosomes of the hepatocytes, either in vitro or in vivo for example, by homologous recombination. Suitable vectors for transforming hepatocytes are well known in the art, and include, for example, various promoters operable in eukaryotic cells, including inducible promoters such as the metallothionein promoter or constitutive promoters such as the SV40 viral promoter, or hepatic-specific promoters such as that associated with α1-antitrypsin. Control elements derived from murine viruses, such as MMTV could also be employed. A particularly useful vector is the plasmid pBRTM/HCV 827-3011 which encodes the HCV NS proteins under control of the T7 promoter and encephalomyocarditis internal ribosome entry site control elements and which directs the synthesis and processing of several HCV non-structural proteins. Insertion of nucleotide sequence encoding the essential hepatic protein into this vector would provide a bicistronic expression system for use in the method of the invention. The T7 promoter could be substituted with other control elements operable in rodent hepatocytes, or hepatocytes modified to contain the pBRTM/HCV vector are further modified to contain an expression system for T7 RNA polymerase. Alternatively, RNA based replicons can be generated by in vitro transcription with T7 RNA polymerase of linearized plasmids, followed by DNase treatment and purification.

In one preferred embodiment, either the DNA or RNA vectors are electroporated into rodent hepatocytes for transplantation. Liposomal-mediated transfection, or other suitable transfection method can also be used. The hepatocytes are first rendered deficient in the essential hepatic protein or otherwise susceptible to selection by the rescue protein(s) and selection for successfully modified cells can be made in in vitro culture under conditions where only the successfully transformed hepatocytes survive. Enhancement of replicon replication efficiency can be achieved by serial cycles of selection. This is achieved as described by Blight, et al., by isolating replicons from transfected cells which grow particularly well under selection, determining the difference in sequence from input replicon, and replacing the mutation(s) associated with the increased replication efficiency back into the next cycle of replicons. Alternatively, the vectors can be directly transfected into the hepatocytes of the recipient rodent in vivo, such as by hydrodynamic transfection.

If in vitro transfection of rodent hepatocytes is used, in the subsequent steps for construction of the model, hepatocytes which have been modified to contain the expression systems for HCV replicons or the replicons themselves each coupled to the selectable marker, are transplanted into rodents whose genetic deficiency corresponds to the rescue protein such as an essential hepatic protein. Techniques for such transplantation are well known in the art. The surviving transplanted rodents then serve as model for HCV infection.

The rodents can then be used to monitor the course of HCV infection per se, to test the effect of either prophylactic or therapeutic treatment of various agents with regard to HCV, to monitor the stage of infection at which anti-HCV therapies act, and to evaluate the efficacy of candidate therapies and prophylactic treatments. The course of infection and assessment of replication can be observed in these model systems and assessed by a variety of means. These include measuring the level of HCV RNA, the level of rescue protein, the level of viral proteins, the level of reporter protein, and the like. The ability of candidate substances to arrest the infection can be assessed by standard methods as well, most simply by observing the amelioration of symptoms when candidate substances or protocols are administered, or monitoring the decrease in replicon RNA, replicon-encoded proteins, rescue proteins or associated reporter proteins or activities. More nuanced evaluations can be obtained by assessing direct effects on liver cells, histological techniques, and the like. Assessment of prophylactic effects can be tested by administering the protocols or compounds designed to exert a protective effect prior to transplanting the hepatocytes.

In those embodiments wherein the deficiency in the rodent can be controlled by administering drugs, an additional advantage is that the efficacy of a treatment can be titrated by partially compensating for the defect at various levels using the defect-reversing drug. For example, in the FAH-deficient murine model, using either NTBC or homogentisic acid, the selective pressure in the FAH mutant mice can be turned off or on and varied in intensity, thus permitting titrating the timing and strength of selection. This is controlled both by timing and dosage.

When appropriate compounds, for instance, are identified, and verified as effective, these can be formulated using standard techniques and used in treatment according to conventional methods of administration.

The following examples are intended to illustrate but not to limit the invention. Murine models are described for convenience, but the methods of the invention are also applicable to other rodents, such as rats.

### EXAMPLE 1

### Preparation of a Bicistronic Vector

The subgenomic bicistronic RNA replicons of HCV described by Blight, K. J., et al., Science (2001) 290:1972-1974 were modified to replace the neomycin resistance gene with the gene encoding FAH. The construct was made using the nucleotide sequence described by Grompe, M., et al., Genes Dev. (1993) 7:2298-2307. cDNA for the functional FAH gene, both mouse and human, were obtained from Dr. Markus Grompe. Standard cloning techniques were employed as previously described by Elazar, M, et al., (2002, in press) and by Glenn, J. S., et al., J. Virol. (1991) 65:2357-2361. The coding sequences were amplified using primers containing unique restriction enzyme sites and the 5′ and 3′ ends of the FAH cDNA coding sequences. Vectors containing reverse transcripts of the Blight, et al replicons were used for these manipulations. To obtain RNA counterparts, in vitro transcription under control of the T7 promoter was employed. Additional replicons were constructed wherein the FAH-encoding sequence was fused to the coding sequence for either luciferase or GFP. A diagrammatic representation of the vectors constructed is shown in FIGS. 2B and 2C; the vector employed by Blight, et al., is shown in FIG. 2A. The resulting vector, designated HCV-FAH comprises the 5′non-coding region of HCV required for replication coupled to the HCV IRES, the initial coding sequence of the core protein fused to the coding sequence for FAH followed by the encephalomyocarditis internal ribosome entry site and a series of nonstructural proteins from HCV: NS3, 4A, 4B, 5A and 5B followed by the 3′non-coding region of HCV required for replication. Alternatively, all the known HCV proteins can be included to generate a full-length HCV replicon.

### EXAMPLE 2

### Transfection of Hepatocytes

The constructs of Example 1 were electroporated or transfected into hepatocyte cells isolated from FAH knockout mouse liver. The hepatocytes were isolated by standard collagenase profusion as described by Overturf, K., et al., M. J. Pathol. (1997) 151:1273-1280, plated on a supportive stromal cell layer of cells as described by Talbot, N. C., et al., In Vitro Cell Dev. Biol. Anim. (1994) 30A:843-850. Cells are cultured on medium containing the substrate for FAH to select cells successfully transformed.

Alternatively, the constructs described can be microinjected into hepatocytes or delivered directly into the liver of appropriate recipient mice, such as by hydrodynamic transfection.

In a preliminary experiment, to determine if the adaptive mutations of Blight, et al., which increase replication efficiency in Huh-7 cells are equally functional in mouse primary hepatocytes, the high efficiency HCV replicon of Blight (containing the neomycin marker) is transfected into primary murine hepatocytes isolated as described above by electroporation. The hepatocytes are then selected in G418. If transformation efficiency is high, the vectors of Blight, et al., are used as described above; if there is insufficient efficiency of colony formation, the colonies which do form are extracted and the RNA amplified by reverse transcription PCR as described by Blight, et al., and incorporated by reference, and sequenced to determine the adaptive mutations optimizing growth in host rodent cells. The isolated mutated vectors are then used as host vectors for replacing the neomycin nucleotide sequence by that encoding FAH or the vectors constructed as above or are modified to contain the desired adaptive mutations identified according to the sequencing results.

### EXAMPLE 3

### Transplantation into Mice

The FAH knockout mice described by Grompe, et al., supra, are used as hosts. The mice are maintained on NTBC (˜7 mg/liter in drinking water) up to an age of 1 month which is acceptable to perform transplantation. The transplantation is performed according to the splenic injection procedure of Overturf, K., et al., Nature Genet. (1996) 12:266-273), and NTBC dosage is withdrawn. Successfully transformed mice survive; mice which do not accommodate the transplanted hepatocytes die within 4-6 weeks. Alternatively, replicons can be introduced into stem cells derived from the bone marrow of FAH knockout mice and these replicon-harboring stem cells are then transplanted into recipient FAH knockout mice leading to hepatic engraftment via stem cell-derived repopulation, or engrafted hepatocytes efficiently supporting high numbers of replicons can be serially transplanted into new recipient mice. In still another alternative, direct in vivo delivery of the replicons or their reverse transcripts can be employed using hydrodynamic transfection, a technique in which nucleic acids are injected into the mouse tail vein under high pressure, thus achieving efficient and largely hepatic-specific delivery. This is described by Liu, F., et al., Gene Therapy (1999) 6:1258-1266; Chang, J., et al., J. Virol. (2001) 75:3469-3473; Bordier, B. B., et al., (2002, manuscript in preparation).

Successful growth of replicon-harboring hepatocytes is monitored by mouse weight gain/survival, immunohistochemical analysis for FAH and/or HCV NS proteins, detection of HCV NS proteins, detection of HCV RNA by Northern blot (Guo, J. T., et al., J. Virol (2001) 75:8516-8523 or by Taqman PCR as described by Blight, et al., (supra). In vivo detection of replication may also employ reporter constructs wherein luciferase or GFP production is monitored using whole-body imaging. In the event whole HCV replicons are used, viremia may also be employed as an assay.

### EXAMPLE 4

### Testing of Candidate HCV Treatments

Successfully transplanted mice as prepared in Example 3 are treated with compounds such as interferon, ribavirin, inhibitors of the amphipathic helices in NS4B or NS5A, or other candidate anti-HCV agents. This is performed in the presence of NTBC for FAH deficient mice, or absence of fructose or galactose in the case of fructose-1-phosphate aldolase or galactose-1-Puridyl transferase deficient mice, respectively. Treated mice show the progress of the disease curtailed as compared to untreated mice, or a decrease in HCV markers such as RNA or specific protein levels. Alternatively, a decrease in reporter level can be measured. If the reporter is luciferase, the treated mice can be imaged in vivo using appropriate equipment and luciferase substrate, and the decrease in HCV replication is reflected in the quantitatively measured decrease in emitted light. If the reporter is a secreted molecule, a decrease in HCV replication in the treated mice can be measured by the level of reporter in biologic samples of the mice (such as blood, urine, feces, etc.).

1. A method to generate a rodent model for HCV infection which method comprises:
introducing into a rodent which harbors a lethal liver-specific defect, using hydrodynamic transfection, an expression system comprising an HCV replicon or a reverse transcript thereof, wherein the HCV replicon or the reverse transcript is coupled to a nucleotide sequence or its complement encoding at least one rescue protein which effects rescue of said liver-specific defect,
wherein said HCV replicon or a reverse transcript thereof is a nucleotide sequence that self-replicates using the replication signals of HCV and encodes a sufficient portion of a HCV genome to recapitulate at least some aspects of HCV infection,
wherein said nucleotide sequences or its complement encoding at least one rescue protein are operably linked to HCV control sequences which effect expression in rodent hepatocytes,
wherein a thus-modified rodent into which said expression system has been introduced is rescued from said liver-specific defect; and
growing said modified rodent, whereby said modified rodent sustains HCV replication and thereby generates a rodent model for HCV infection.

2. The method of claim 1 wherein the HCV replicon or reverse transcript contains nucleotide sequences corresponding to less than the complete HCV virion.

3. The method of claim 1 wherein said HCV replicon or reverse transcript contains nucleotide sequences corresponding to the complete HCV virion.

4. The method of claim 1 wherein said expression system is adapted for efficient replication in rodent hepatocytes.

5. The method of claim 1 wherein said liver-specific defect is the lack of an essential hepatic protein, and said rescue protein is an essential hepatic protein.

6. The method of claim 5 wherein said essential hepatic protein is fumarylacetoacetate hydrolase (FAH).

7. The method of claim 1 wherein the expression system further comprises a nucleotide sequence or its complement encoding a reporter protein coupled to said HCV replicon.

8. The method of claim 7 wherein the reporter protein is luciferase or green fluorescent protein (GFP).

9. A rodent model for HCV infection which is prepared by the method of claim 1.

10. A rodent model for HCV infection which is prepared by the method of claim 7.

11. A method to generate a rodent model for HCV infection which method comprises: introducing into a rodent which harbors a lethal liver-specific defect an expression system comprising an HCV replicon or a reverse transcript thereof, wherein the HCV replicon or the reverse transcript is coupled to a nucleotide sequence or its complement encoding at least one rescue protein which effects rescue of said liver-specific defect by transplanting hepatocytes of a rodent species which have been modified to contain said expression system in in vitro culture,
wherein said HCV replicon or a reverse transcript thereof is a nucleotide sequence that self-replicates using the replication signals of HCV and encodes a sufficient portion of a HCV genome to recapitulate at least some aspects of HCV infection,
wherein said nucleotide sequences or its complement encoding at least one rescue protein are operably linked to HCV control sequences which effect expression in said rodent hepatocytes,
wherein a thus-modified rodent into which said expression system has been introduced is rescued from said liver-specific defect; and
growing said modified rodent, whereby said modified rodent sustains HCV replication and thereby generates a rodent model for HCV infection.

12. The method of claim 11 wherein the HCV replicon or reverse transcript contains nucleotide sequences corresponding to less than the complete HCV virion.

13. The method of claim 1 1 wherein said HCV replicon or reverse transcript contains nucleotide sequences corresponding to the complete HCV virion.

14. The method of claim 11 wherein said expression system is adapted for efficient replication in rodent hepatocytes.

15. The method of claim 1I1 wherein said liver-specific defect is the lack of an essential hepatic protein, and said rescue protein is an essential hepatic protein.

16. The method of claim 15 wherein said essential hepatic protein is fumarylacetoacetate hydrolase (FAH).

17. The method of claim 11 wherein the expression system further comprises a nucleotide sequence or its complement encoding a reporter protein coupled to said HCV virion.

18. The method of claim 17 wherein the reporter protein is luciferase or green fluorescent protein (GFP).

19. A rodent model for HCV infection which is prepared by the method of claim 11.

20. A rodent model for HCV infection which is prepared by the method of claim 17.
